(19) United States
(12) Patent Application Publication
(10) Pub. No.: US 2024/0280534 A1
(43) Pub. Date: Aug. 22, 2024

(54) SENSOR AND METHOD FOR DETECTING BIOLOGICAL SUBSTANCE

(71) Applicants: MITSUBISHI MATERIALS CORPORATION, Tokyo (JP); JAPAN ADVANCED INSTITUTE OF SCIENCE AND TECHNOLOGY, Nomi-shi (JP)

(72) Inventors: Toshihiro Doi, Naka-shi (JP); Yuzuru Takamura, Nomi-shi (JP); Daisuke Hirose, Nomi-shi (JP)

(73) Assignees: MITSUBISHI MATERIALS CORPORATION, Tokyo (JP); JAPAN ADVANCED INSTITUTE OF SCIENCE AND TECHNOLOGY, Nomi-shi (JP)

(21) Appl. No.: 18/569,734

(22) PCT Filed: Jun. 17, 2022
(86) PCT No.: PCT/JP2022/024363

§ 371 Date: Dec. 13, 2023

(30) Foreign Application Priority Data
Jun. 18, 2021 (JP) 2021-101728

(51) Int. Cl. G01N 27/414; G01N 27/327; G01N 27/333

(52) U.S. Cl. CPC G01N 27/4145; G01N 27/327; G01N 27/333

(57) ABSTRACT

A sensor includes a first electrode, a second electrode, a third electrode, a semiconductor film that connects the first electrode and the second electrode, and a solid electrolyte membrane that covers the first electrode, the second electrode, and the semiconductor film, in which the solid electrolyte membrane has an exposed surface exposed to an outside, and the third electrode is configured to be disposed at a position where when the exposed surface of the solid electrolyte membrane is in contact with a conductive liquid (Lq), it is possible to apply an electric field to the exposed surface of the solid electrolyte membrane through the conductive liquid (Lq).

### TECHNICAL FIELD

The present invention relates to a sensor and a method for detecting a biological substance.

Priority is claimed on Japanese Patent Application No. 2021-101728, filed Jun. 18, 2021, the content of which is incorporated herein by reference.

### BACKGROUND ART

As methods for detecting a biological substance from a test liquid including biological substances such as mRNA and DNA, a polymerase chain reaction (PCR) method and next generation sequencing are known. The PCR method is a method in which a specific region (target region) on a DNA sequence is amplified by using a heat-resistant DNA polymerase, and since it is possible to detect from a single DNA molecule, a specific sequence of DNA can be detected with high sensitivity. In addition, the next generation sequencing is a method of fragmenting DNA to prepare a library and sequencing the DNA fragments of the library in parallel, and the method enables comprehensive decoding of an entire DNA sequence from a single molecule. In these methods for detecting a biological substance, a detection time is required.

As a method for detecting a biological substance such as DNA in a short period of time, using a sensor having a thin film transistor (TFT) structure has been studied.

For example, a method has been reported in which a homo-oligomeric DNA strand is immobilized by using 3-aminopropylethoxysilane, and hybridization with the above-described oligomeric strand is directly detected by displacement of the gate potential under a constant drain current (Non-Patent Document 1). Non-Patent Document 1 discloses a sensor having a thin film transistor structure in which a source electrode, a drain electrode, and a channel are covered with a dielectric.

### CITATION LIST

### Non-Patent Document

[Non-Patent Document 1]
   J. Phys. Chem. B, 1997, 101, 2980-2985.

### SUMMARY OF INVENTION

### Technical Problem

With regard to a sensor for detecting a biological substance, a test liquid that is used as a sample is a mixture (liquid) in which a biological substance other than the biological substance intended to be detected is co-present. For this reason, it is desirable that a sensor for detecting a biological substance has excellent selectivity for a biological substance intended to be detected, has high sensitivity, and has a low detection limit. However, the sensor having a thin film transistor structure described in Non-Patent Document 1 has a detection limit of about 1 μg/mL, and further improvement of the sensitivity is desired. In order to improve the sensitivity of a sensor having a thin film transistor structure, it is conceivable to remove the dielectric to expose the source electrode, the drain electrode, and the channel. However, when the source electrode or the drain electrode is exposed, there is a risk that a current may flow directly from the solution to the source electrode and the drain electrode (leakage current) at the time of measurement, and due to a pH change or the like resulting from an electrochemical reaction associated with that leakage current, obtained data may be destabilized. In addition, when it is attempted to expose only the channel portion, there is a risk that the test liquid may infiltrate at the interface between the source electrode as well as the drain electrode and the channel, which may become new instability factors.

The present invention was made in view of the above-described circumstances, and an objective of the present invention is to provide a sensor having excellent selectivity for a detection target substance and having high sensitivity and a low detection limit, and a method for detecting a biological substance.

### Solution to Problem

The inventors of the present invention conducted thorough studies, and as a result, the inventors found that in a sensor having a thin film transistor structure having a first electrode, a second electrode, a third electrode, and a semiconductor film connecting the first electrode and the second electrode, when the first electrode, the second electrode, and the semiconductor film are covered with a solid electrolyte membrane, and an exposed surface of the solid electrolyte membrane exposed to an outside is in contact with a conductive liquid, by configuring the sensor such that the third electrode is disposed at a position where it is possible to apply an electric field to the exposed surface of the solid electrolyte film through the conductive liquid, the generation of leakage current can be suppressed, and a detection target substance can be detected with excellent selectivity, high sensitivity, and a low detection limit, thereby completing the present invention.

That is, the present invention provides the following means to solve the above-described problems.

[1] A sensor including:
a first electrode;
a second electrode;
a third electrode;
a semiconductor film configured to connect the first electrode and the second electrode; and
a solid electrolyte membrane configured to cover the first electrode, the second electrode, and the semiconductor film,
in which the solid electrolyte membrane has an exposed surface exposed to an outside, and
the third electrode is configured to be disposed at a position where, when the exposed surface of the solid electrolyte membrane is in contact with a conductive liquid, it is possible to apply an electric field to the exposed surface of the solid electrolyte membrane through the conductive liquid.

[2] The sensor according to [1], in which the first electrode, the second electrode, and the semiconductor film are arranged on one substrate.

[3] The sensor according to [2], in which a conductive material film and a solid electrolyte film are laminated between the substrate and the first electrode, the second electrode, as well as the semiconductor film, and
the first electrode, the second electrode, and the semiconductor film are arranged on the solid electrolyte film.

[4] The sensor according to [2] or [3], in which the third electrode is further disposed on the substrate.

[5] The sensor according to any one of [1] to [4], in which the solid electrolyte membrane has an ion conductivity of 1×10⁻⁸ S/cm or more.

[6] The sensor according to any one of [1] to [5], in which the solid electrolyte membrane is an inorganic solid electrolyte membrane which is a metal oxide including a rare earth element and zirconium (Zr) or a metal oxide including a rare earth element and tantalum (Ta) and in which the amount of carbon (C) is 0.5 atom % or more and 15 atom % or less while the amount of hydrogen (H) is 2 atom % or more and 20 atom % or less, and the semiconductor film is an inorganic semiconductor film which is a metal oxide including at least indium (In).

[7] The sensor according to any one of [1] to [6], in which probe molecules intended for capturing a biological substance are immobilized on the exposed surface of the solid electrolyte membrane.

[8] The sensor according to any one of [1] to [7], further including a holding part intended for holding the conductive liquid around the exposed surface of the solid electrolyte membrane.

[9] A method for detecting a biological substance using the sensor according to any one of [1] to [8], the method including:
a step of supplying a test liquid including a biological substance to the exposed surface of the solid electrolyte membrane to capture the biological substance to the exposed surface;
a step of replacing the test liquid with the conductive liquid;
a step of applying a voltage between the third electrode and the first electrode, and at the same time, measuring a current between the first electrode and the second electrode; and
a step of acquiring an amount of the biological substance in the test liquid based on the voltage and the current.

### Advantageous Effects of Invention

According to the present invention, it is possible to provide a sensor that has excellent selectivity to a detection target substance and has high sensitivity and a low detection limit, and a method for detecting a biological substance.

### BRIEF DESCRIPTION OF DRAWINGS

FIG. 1 is a plan view showing an example of a sensor according to an embodiment of the present invention.

FIG. 2 is a cross-sectional view taken along line II-II′ in FIG. 1.

FIG. 3A is a schematic view showing a method for detecting a biological substance using the sensor shown in FIG. 1, and is an enlarged cross-sectional view of the sensor as viewed along line II-II′ in FIG. 1.

FIG. 3B is an enlarged view of FIG. 3A.

FIG. 4 is a plan view showing another example of the sensor according to an embodiment of the present invention.

FIG. 5A is a schematic view showing a method for detecting a biological substance using the sensor shown in FIG. 4, and is a cross-sectional view of the sensor as viewed along line V-V′ in FIG. 4.

FIG. 5B is an enlarged view of FIG. 5A.

FIG. 6A is V_{TG}-I_{G} curves measured in Invention Example 1 and Comparative Examples 1 and 2.

FIG. 6B is V_{TG}-I_{SD} curves measured in Invention Example 1 and Comparative Examples 1 and 2.

FIG. 7 is a flowchart showing a procedure for immobilizing a probe DNA on a holding part of a sensor in Invention Example 2.

FIG. 8 is a graph showing a V_{TG}-I_{SD} curve measured in Invention Example 2.

FIG. 9 is a graph showing a V_{TG}-I_{SD} curve measured in Invention Example 3.

### DESCRIPTION OF EMBODIMENTS

Hereinafter, a sensor and a method for detecting a biological substance, which constitute an embodiment of the present invention, will be described in detail with reference to the drawings. In the drawings used in the following description, characteristic portions may be enlarged for convenience in order to make the characteristics of the present embodiment easier to understand, and the dimensional ratio and the like of each constituent element may be different from the actual ones.

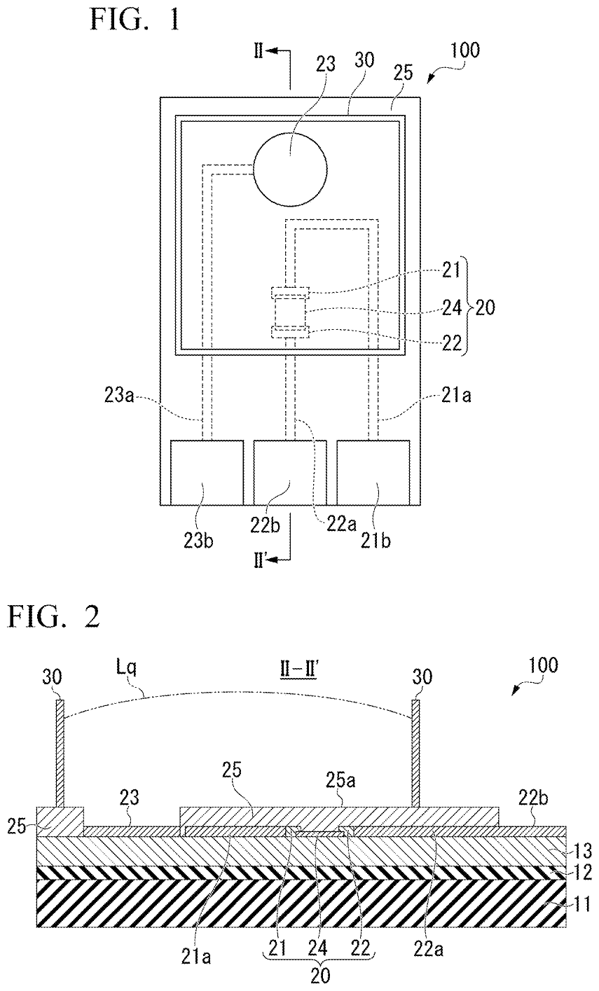

FIG. 1 is a plan view showing an example of a sensor according to an embodiment of the present invention, and FIG. 2 is a cross-sectional view taken along line II-II′ of FIG. 1.

As shown in FIG. 1 and FIG. 2, a sensor 100 has a substrate 11, a first electrode 21, a second electrode 22, a third electrode 23, a semiconductor film 24, and a solid electrolyte membrane 25. The semiconductor film 24 is disposed at a position that connects the first electrode 21 and the second electrode 22. The first electrode 21, the second electrode 22, and the semiconductor film 24 form a sensor piece 20. A conductive material film 12 and a solid electrolyte film 13 are laminated between the sensor piece 20 (first electrode 21, second electrode 22, and semiconductor film 24) as well as the third electrode 23 and the substrate 11, and the sensor piece 20 and the third electrode 23 are disposed on the solid electrolyte film 13. The solid electrolyte membrane 25 covers the sensor piece 20. The solid electrolyte membrane 25 has an exposed surface 25a exposed to the outside (upper side, that is, on the opposite side of the sensor piece 20). The third electrode 23 is configured to be disposed at a position where when the exposed surface 25a of the solid electrolyte membrane 25 is in contact with a conductive liquid Lq, it is possible to apply an electric field to the exposed surface 25a of the solid electrolyte membrane 25 through the conductive liquid Lq. That is, the third electrode 23 is disposed to be electrically connected to the exposed surface 25a of the solid electrolyte membrane 25 through the supplied conductive liquid Lq. The conductive liquid Lq is a liquid having electrical conductivity so that an electric field can be applied from the third electrode 23 to the exposed surface 25a. As the conductive liquid Lq, for example, an aqueous solution including an inorganic salt can be used. The inorganic salt may be any substance which is substantially inert during use with respect to the detection target substance and the solid electrolyte membrane 25, and there is no particular limitation thereof.

It is preferable that the exposed surface 25a of the solid electrolyte membrane 25 is at a position facing the semiconductor film 24. It is preferable that the exposed surface 25a has immobilized thereon a capturing substance for capturing the detection target substance. For example, when the detection target substance is a biological substance, probe molecules for capturing the biological substance may be immobilized. The exposed surface 25a and the third electrode 23 are surrounded by a holding part 30 for holding the conductive liquid Lq. The shape of the holding part 30 is not limited; however, the shape is preferably, for example, a wall shape as shown in FIGS. 1 and 2.

The first electrode 21 is connected to a first terminal 21b through a first lead wire 21a. The second electrode 22 is connected to a second terminal 22b through a second lead wire 22a. The third electrode 23 is connected to a third terminal 23b through the third lead wire 23a.

As the material for the first electrode 21, the second electrode 22, and the third electrode 23, a metal material and a metal oxide can be used. Examples of the metal material include high-melting point metals such as platinum (Pt), and alloys thereof. Examples of the metal oxide include indium tin oxide (ITO) and ruthenium oxide (RuO₂). Each of the first electrode 21, the second electrode 22, and the third electrode 23 may be a single layer body or may be a multilayer body in which a plurality of electrode material layers are laminated. The thicknesses of the first electrode 21, the second electrode 22, and the third electrode 23 may be in the range of, for example, 50 nm or more and 200 nm or less. The first lead wire 21a and the first terminal 21b may be made of the same material as the first electrode 21 and may have the same thickness as the first electrode 21. The second lead wire 22a and the second terminal 22b may be made of the same material as the second electrode 22 and may have the same thickness as the second electrode 22. The third lead wire 23a and the third terminal 23b may be made of the same material as the third electrode 23 and may have the same thickness as the third electrode 23.

The semiconductor film 24 may be an inorganic semiconductor film or an organic semiconductor film.

The inorganic semiconductor film includes an inorganic semiconductor. It is preferable that the inorganic semiconductor film is formed only of an inorganic semiconductor. It is preferable that the inorganic semiconductor includes at least one inorganic substance selected from the group consisting of, for example, indium oxide (In₂O₃), zinc oxide (ZnO), In—Ga—Zn oxide (IGZO), In—Sn—Zn oxide (ITZO), Zn—Sn oxide (Zn—Sn—O), amorphous silicon (a-Si), low-temperature polysilicon (LTPS), and graphene. Regarding these inorganic semiconductors, one kind thereof may be used alone, or two kinds thereof may be used in combination. In addition, the inorganic semiconductor may be in an amorphous phase or a nanocrystalline phase.

The organic semiconductor film includes an organic semiconductor. It is preferable that the organic semiconductor film is formed only of an organic semiconductor. It is preferable that the organic semiconductor is a polycyclic aromatic hydrocarbon or a thienoacene-based compound.

It is preferable that the polycyclic aromatic hydrocarbon has four or more benzene rings. The polycyclic aromatic hydrocarbon is preferably an acene. The acene may have a substituent (for example, a phenyl group). Examples of the polycyclic aromatic hydrocarbon include pentacene and rubrene. Examples of the thienoacene-based compound include BTBT, DNTT, C8-DNTT, and C10-DNBOT. Regarding these organic semiconductors, one kind thereof may be used alone, or two kinds thereof may be used in combination. In addition, the organic semiconductor may be in an amorphous phase or a nanocrystalline phase.

The semiconductor film 24 may be a single layer body or may be a multilayer body in which a plurality of semiconductor layers are laminated. The thickness of the semiconductor film 24 may be in the range of, for example, 5 nm or more and 80 nm or less. The length of the semiconductor film 24 (distance between the first electrode 21 and the second electrode 22) is, for example, 50 μm or more and 200 μm or less. The width of the semiconductor film 24 (length of contact with the first electrode 21 and the second electrode 22) may be in the range of, for example, 1 μm or more and 10,000 μm or less.

The solid electrolyte membrane 25 may be proton-conductive. The solid electrolyte membrane 25 may have an ion conductivity of 1×10⁻⁸ S/cm or more. The ion conductivity of the solid electrolyte membrane 25 may be 1×10⁻² S/cm or less.

The solid electrolyte membrane 25 may be an inorganic solid electrolyte membrane or an organic solid electrolyte membrane.

The inorganic solid electrolyte membrane includes an inorganic solid electrolyte. It is preferable that the inorganic solid electrolyte membrane is formed only of an inorganic solid electrolyte. For example, the inorganic solid electrolyte membrane can be formed of either a metal oxide including a rare earth element and zirconium (Zr), or a metal oxide including a rare earth element and tantalum (Ta). The amount of carbon (C) in the inorganic solid electrolyte membrane may be in the range of 0.5 atom % or more and 15 atom % or less. Further, the amount of hydrogen (H) in the inorganic solid electrolyte membrane may be in the range of 2 atom % or more and 20 atom % or less. When the inorganic solid electrolyte membrane is formed of the above-described metal oxide, and both the amounts of carbon (C) and hydrogen (H) are within the above-described ranges, the sensor 100 acquires high sensitivity, the detection limit is lowered to a large extent, and the detection stability in the presence of moisture or the like is increased. From the viewpoint of further improving these characteristics, the amount of carbon (C) may be set in the range of 1 atom % or more and 10 atom % or less, and the amount of hydrogen (H) may be set in the range of 5 atom % or more and 18 atom % or less.

The inorganic solid electrolyte membrane can be formed by, for example, any of the following (A1) to (A5).

(A1) A metal oxide including lanthanum (La) and zirconium (Zr)

(A2) A metal oxide including lanthanum (La) and tantalum (Ta)

(A3) A metal oxide including any metal element selected from the group consisting of cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), lutetium (Lu), and yttrium (Y), and zirconium (Zr) or tantalum (Ta)

(A4) A metal oxide including at least one kind of metal element selected from the group consisting of hafnium (Hf), zirconium (Zr), and aluminum (Al)

(A5) A metal oxide containing lanthanum (La), hafnium (Hf), or zirconium (Zr)

For example, when the inorganic solid electrolyte membrane is formed of a metal oxide including lanthanum (La) and zirconium (Zr) (A1), the atomic ratio between lanthanum (La) and zirconium (Zr) is such that, for example, when lanthanum (La) is set to 1, zirconium (Zr) may be in the range of 0.43 or more and 2.33 or less or may be in the range of 1.00 or more and 2.33 or less. In addition, when the inorganic solid electrolyte membrane is formed of a metal oxide including lanthanum (La) and tantalum (Ta) (A2), the atomic ratio between lanthanum (La) and tantalum (Ta) is also not particularly limited. Furthermore, when the inorganic solid electrolyte membrane is formed of any of the metal oxides of (A3) to (A5) described above, the atomic ratio of each metal element is also not particularly limited. The inorganic solid electrolyte may be in an amorphous phase.

The atomic composition ratios of the metal oxides can be determined by performing elemental analysis using Rutherford backscattering spectrometry (RBS method) or the like. In addition, the amounts of carbon (C) and hydrogen (H) can be determined by performing elemental analysis by using Rutherford backscattering spectrometry (RBS analysis method), hydrogen forward scattering spectrometry (HFS analysis method), and nuclear reaction analysis (NRA analysis method).

The organic solid electrolyte membrane includes an organic solid electrolyte. It is preferable that the organic solid electrolyte membrane is formed only of an organic solid electrolyte. It is preferable that the organic solid electrolyte is, for example, proton-conductive. As the organic solid electrolyte, for example, a polymer having a proton-conductive group in a side chain, or an organic metal complex may be used.

The main chain of the polymer having the proton-conductive group in a side chain may have, for example, a hydrocarbon structure or a perfluorocarbon structure. The proton-conductive group may be, for example, a sulfonic acid group. As the polymer having a proton-conductive group in a side chain, NAFION (registered trademark), which is a perfluorocarbon sulfonic acid, can be used.

The organic metal complex may be, for example, a coordination polymer. The coordination polymer may be an oxalate crosslinked coordination polymer represented by the following Formula (1).

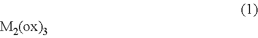

In (1) as described above, M represents a divalent or trivalent metal ion. When M is a trivalent metal ion, the oxalate crosslinked coordination polymer is neutral. When M includes a divalent metal ion, the oxalate crosslinked coordination polymer is anionic, and a counterion may be incorporated into the oxalate crosslinked coordination polymer.

In (1) described above, ox represents an oxalate ion (C₂O₄²⁻).

Regarding the organic solid electrolyte, one kind thereof may be used alone, or two kinds thereof may be used in combination. In addition, the organic solid electrolyte may be in an amorphous phase or a nanocrystalline phase.

The solid electrolyte membrane 25 may be a single layer body or may be a multilayer body in which a plurality of solid electrolyte layers are laminated. The thickness of the solid electrolyte membrane 25 may be in the range of, for example, 1 nm or more and 100 nm or less.

As the substrate 11, for example, an insulating substrate and a semiconductor substrate can be used. Examples of the insulating substrate include high heat-resistant glass, an alumina (Al₂O₃) substrate, an STO (SrTiO) substrate, a SiO₂/Si substrate (obtained by forming a SiO₂ film on a Si substrate), and a multilayer substrate in which an STO (SrTiO) layer is formed on a surface of an Si substrate, with a SiO₂ layer and a Ti layer interposed therebetween. Examples of the semiconductor substrate include a Si substrate, a SiC substrate, and a Ge substrate. The thickness of the substrate 11 is, for example, 10 μm or more and 1 mm or less.

The conductive material film 12 is a conductive material film including a conductive material. The conductive material film 12 may be formed only of a conductive material. As the conductive material, for example, a metal material and a metal oxide can be used. Examples of the metal material include platinum (Pt), gold (Au), silver (Ag), copper (Cu), aluminum (Al), molybdenum (Mo), palladium (Pd), ruthenium (Ru), iridium (Ir), tungsten (W), titanium (Ti), and alloys of these metals. Examples of the metal oxide include indium tin oxide (ITO) and ruthenium oxide (RuO₂).

The conductive material film 12 may be a single layer body or a multilayer body in which a plurality of conductive material films are laminated. The thickness of the conductive material film 12 is, for example, 50 nm or more and 200 nm or less.

The solid electrolyte film 13 may be proton-conductive. The solid electrolyte film 13 may have an ion conductivity of 1×10⁻⁸ S/cm or more. The ion conductivity of the solid electrolyte film 13 may be 1×10⁻² S/cm or less. The solid electrolyte film 13 may be an inorganic solid electrolyte film or an organic solid electrolyte film. Examples of the materials of the inorganic solid electrolyte film and the organic solid electrolyte film are the same as in the case of the solid electrolyte membrane 25.

The solid electrolyte film 13 may be a single layer body or may be a multilayer body in which a plurality of solid electrolyte layers are laminated. The thickness of the solid electrolyte film 13 may be in the range of, for example, 50 nm or more and 300 nm or less.

The material of the holding part 30 may be an organic substance or may be an inorganic substance. Examples of the organic substance include polyimide and an epoxy resin. Examples of the inorganic substance include alumina and silica. The height of the holding part 30 may be, for example, in the range of 0.10 mm or more and 5 mm or less. The thickness of the holding part 30 may be, for example, in the range of 0.5 mm or more and 5 mm or less and is preferably about 1 mm.

Next, a method for detecting a biological substance using the sensor 100 will be described.

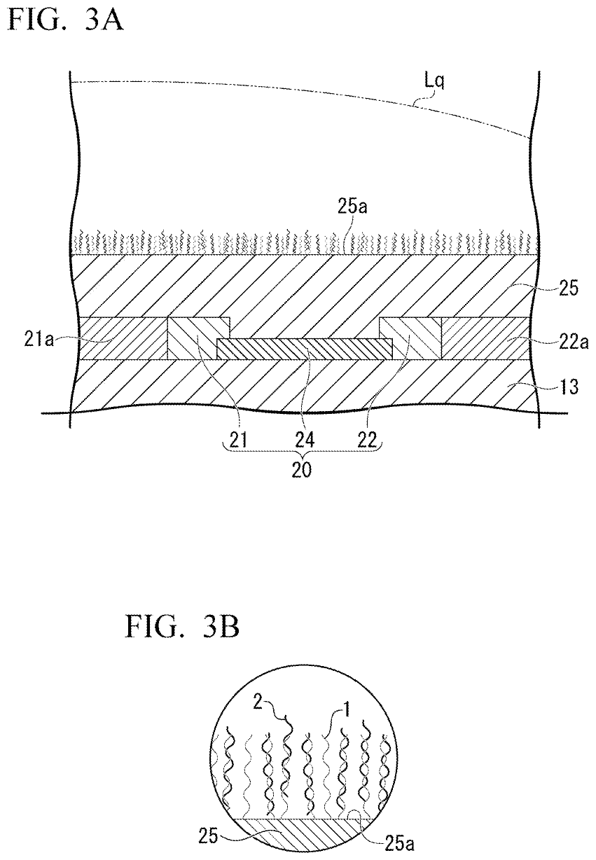

FIG. 3A is a schematic view showing a method for detecting a biological substance using the biological substance detection sensor shown in FIG. 1 and is an enlarged cross-sectional view of the biological substance detection sensor as viewed along line II-II′ in FIG. 1, and FIG. 3B is an enlarged view of FIG. 3A. In FIG. 3B, probe molecules 1 for capturing a biological substance are immobilized on an exposed surface 25a on the inside of the holding part 30 of the sensor 100.

Detection of a biological substance using the sensor 100 is carried out as follows.

First, a biological substance 2 intended for detection is captured by the probe molecules 1 immobilized on the exposed surface 25a of the sensor 100. Specifically, a test liquid including the biological substance 2 is injected into the holding part 30 of the sensor 100 to supply the test liquid to the exposed surface 25a of the solid electrolyte membrane 25. As a result, the biological substance 2 intended for detection is captured to the exposed surface 25a through the probe molecules 1. The biological substance 2 is, for example, a nucleic acid such as DNA or mRNA. The probe molecule 1 is DNA or mRNA that is complementary to a portion of that biological substance 2.

After the biological substance 2 is captured to the exposed surface 25a, the test liquid is replaced with a conductive liquid Lq. Specifically, first, the exposed surface 25a is washed with a cleaning liquid to remove, for example, a biological substance that is not captured by the probe molecules 1 (biological substance not intended for detection) or a biological substance that is non-specifically captured. Next, the conductive liquid Lq is injected into the holding part 30 of the sensor 100 to bring the third electrode 23 and the exposed surface 25a into contact with the conductive liquid Lq. As the conductive liquid Lq, for example, a phosphate buffer (PBS) can be used. As a result, the third electrode 23 can apply an electric field to the exposed surface 25a through a conductive liquid.

Next, a voltage V_{SD} is applied between the first electrode 21 and the second electrode 22, and a voltage V_{TG} is applied between the first electrode 21 and the third electrode 23. In the sensor 100 of the present embodiment, as an electric field created by the charge of the biological substance 2 captured to the exposed surface 25a through the probe molecules 1 is transmitted to the semiconductor film 24, the electrical characteristics of the semiconductor film 24 change. Therefore, when an electric field with a voltage V_{SD} is applied from the third electrode 23 to the exposed surface 25a, a current I_{SD} flowing between the first electrode 21 and the second electrode 22 changes. From this relationship between the voltage V_{SD} and the current I_{SD}, the biological substance 2 captured by the probe molecules 1 can be quantified, and from this, the amount of the biological substance in the test liquid can be acquired.

The sensor 100 can be produced, for example, as follows.

### (1) Deposition of Conductive Material Film 12

First, a conductive material film 12 is deposited on a substrate 11 (for example, a SiO₂/Si substrate). As a method for depositing the conductive material film 12, a sputtering method can be used.

### (2) Deposition of Solid Electrolyte Film 13

Next, a solid electrolyte film 13 is deposited on the conductive material film 12. The solid electrolyte film 13 can be formed by, for example, applying a precursor solution for a solid electrolyte film on the conductive material film 12 and heating the obtained coating film. As the precursor solution for a solid electrolyte film, a liquid in which a material of the solid electrolyte constituting the solid electrolyte film 13 is dissolved or dispersed, can be used. As a method for applying the precursor solution for a solid electrolyte film, for example, a spin coating method, an inkjet printing method, and a nanoimprinting method can be used. The heating temperature for the coating film is not particularly limited as long as it is a temperature at which the solvent of the precursor solution for a solid electrolyte film is volatilized and the solid electrolyte film 13 is generated.

### (3) Deposition of Semiconductor Film 24

Next, a semiconductor film 24 is deposited on the solid electrolyte film 13. The semiconductor film 24 can be deposited, for example, as follows. First, a resist film patterned by a photolithography method is formed on the solid electrolyte film 13. Next, a precursor solution for a semiconductor film is applied on the solid electrolyte film 13 on which the resist film has been formed, and the obtained coating film is heated to form a semiconductor film. Thereafter, the resist film is removed. As the precursor solution for a semiconductor film, a liquid in which a material of the semiconductor constituting the semiconductor film 24 is dissolved or dispersed, can be used. As a method for applying the precursor solution for a semiconductor film, for example, a spin coating method, an inkjet printing method, and a nanoimprinting method can be used. The heating temperature for the coating film is not particularly limited as long as it is a temperature at which the solvent of the precursor solution for a semiconductor film is volatilized and the semiconductor film 24 is generated.

### (4) Formation of Electrode Pattern

Next, an electrode pattern (a first electrode 21, a first lead wire 21a, a first terminal 21b, a second electrode 22, a second lead wire 22a, a second terminal 22b, a third electrode 23, a third lead wire 23a, and a third terminal 23b) is formed on the solid electrolyte film 13 and the semiconductor film 24. The electrode pattern can be formed, for example, as follows. First, a resist film patterned by a photolithography method is formed on the solid electrolyte film 13 and the semiconductor film 24. Next, an electrode film is formed on the solid electrolyte film 13 and the semiconductor film 24, on which the resist film has been formed. Thereafter, the resist film is removed. As a method for forming the electrode film, for example, a sputtering method can be used.

### (5) Deposition of Solid Electrolyte Membrane 25

Next, a solid electrolyte membrane 25 is deposited on a sensor piece 20 (the first electrode 21, the second electrode 22, and the semiconductor film 24). The solid electrolyte membrane 25 can be deposited, for example, as follows. First, a resist film is formed on the first terminal 21b, the second terminal 22b, and the third electrode 23. Next, a precursor solution for a solid electrolyte membrane is applied thereon, and the obtained coating film is heated to form a solid electrolyte membrane. Thereafter, the resist film is removed. As the precursor solution for a solid electrolyte membrane, a liquid in which a material of the solid electrolyte constituting the solid electrolyte membrane 25 is dissolved or dispersed, can be used. As a method for applying the precursor solution for a solid electrolyte membrane, for example, a spin coating method, an inkjet printing method, and a nanoimprinting method can be used. The heating temperature for the coating film is not particularly limited as long as it is a temperature at which the solvent in the precursor solution for a solid electrolyte membrane is volatilized and a solid electrolyte membrane 25 is generated.

In the above-described sensor 100, the third electrode 23 is formed on the solid electrolyte film 13 of the substrate 11; however, the position of the third electrode 23 is not limited to this. The third electrode 23 may be disposed at a position other than on the solid electrolyte film 13 as long as the third electrode 23 is configured to be disposed at a position where when the exposed surface 25a of the solid electrolyte membrane 25 is in contact with the conductive liquid Lq, it is possible to apply an electric field to the exposed surface 25a of the solid electrolyte membrane 25 through the conductive liquid Lq.

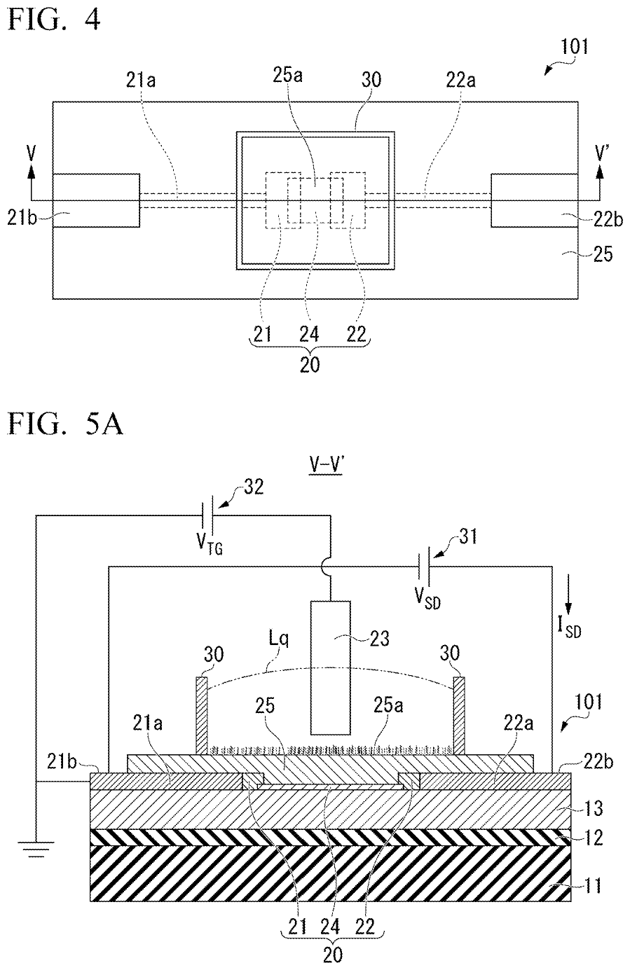

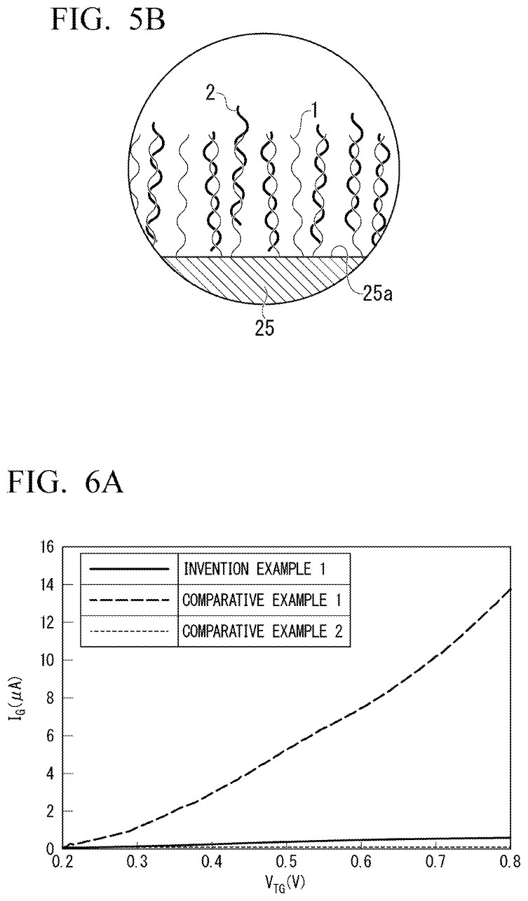

FIG. 4 is a plan view showing another example of the sensor according to an embodiment of the invention, and FIG. 5 is a schematic view showing a method for detecting a biological substance using the sensor shown in FIG. 4, in which FIG. 5A is a cross-sectional view of the sensor as viewed along the line V-V′ in FIG. 4, while FIG. 5B is an enlarged view of FIG. 5A.

A sensor 101 shown in FIG. 4 and FIG. 5 has a substrate 11, a first electrode 21, a second electrode 22, a third electrode 23, a semiconductor film 24, and a solid electrolyte membrane 25. A conductive material film 12 and a solid electrolyte film 13 are laminated between a sensor piece 20 (the first electrode 21, the second electrode 22, and the semiconductor film 24) and the substrate 11, and the third electrode 23 is disposed on the sensor piece 20 and the solid electrolyte film 13. The sensor 101 is configured such that the third electrode 23 is separated from the substrate 11, and a portion of the third electrode 23 is immersed in a conductive liquid Lq held in a holding part 30. Other configurations are the same as the configurations of the above-mentioned sensor 100. Therefore, the same reference numerals will be used for the same or similar portions in the sensor 101 shown in FIG. 4 and FIG. 5 and the above-mentioned sensor 100, and descriptions thereof will not be repeated herein.

Detection of a biological substance using the sensor 101 can be carried out as follows.

First, as in the case of the above-mentioned sensor 100, a test liquid including a biological substance 2 is supplied to an exposed surface 25a of the solid electrolyte membrane 25 to capture the biological substance 2 to the exposed surface 25a by means of probe molecules 1. Next, the test liquid is replaced with the conductive liquid Lq.

A portion of the third electrode 23 is immersed in the conductive liquid Lq that has been injected into a holding part 30 of the sensor 101. As a result, the third electrode 23 can apply an electric field to the exposed surface 25a through a conductive liquid.

The first electrode 21 and the second electrode 22 are connected to a first voltage supply part 31, and the first electrode 21 and the third electrode 23 are connected to a second voltage supply part 32. Quantification of the biological substance 2 captured by the probe molecules 1 can be performed in the same manner as in the case of the sensor 100 shown in FIG. 1 to FIG. 3B. That is, a voltage V_{SD} is applied between the first electrode 21 and the second electrode 22 by using the first voltage supply part 31, and a voltage V_{TG} is applied between the first electrode 21 and the third electrode 23 by using the second voltage supply part 32. As a result, a current I_{SD} flowing between the first electrode 21 and the second electrode 22 changes. Then, from this relationship between the voltage V_{SD} and the current I_{SD}, the biological substance 2 captured by the probe molecules 1 can be quantified, and from this, the amount of the biological substance in the test liquid can be acquired.

According to the sensors 100 and 101 of the present embodiment configured as described above, when the voltage V_{SD} is applied between the first electrode 21 and the second electrode 22 while at the same time, the voltage V_{TG} is applied between the first electrode 21 and the third electrode 23, an electric field is applied to the semiconductor film 24 through the solution and the solid electrolyte, and the current I_{SD} flowing between the first electrode 21 and the second electrode 22 flows. Since the strength of the electric field applied to the semiconductor film 24 varies depending on the amount of the biological substance 2 captured to the exposed surface 25a, according to the sensors 100 and 101 of the present embodiment, the biological substance 2 captured to the exposed surface 25a can be selectively quantified with high sensitivity from the relationship between the voltage V_{SD} and the current I_{SD}. Furthermore, in the sensors 100 and 101 of the present embodiment, since the first electrode 21, the second electrode 22, and the semiconductor film 24 are covered with the solid electrolyte membrane 25, when an electric field is applied from the third electrode 23 to the exposed surface 25a of the solid electrolyte membrane 25 through the test liquid Lq, the generation of leakage current is suppressed. By suppressing the generation of this leakage current, the above-described destabilization caused by the leakage current can be suppressed, and detection can be performed with higher sensitivity and a lower detection limit.

In the sensors 100 and 101 of the present embodiment, since the solid electrolyte membrane 25 is a solid electrolyte, it is possible to induce more charges in the semiconductor film than in a case where the solid electrolyte membrane 25 is formed of an insulator or a dielectric, the current I_{SD} is large, and the mutual conductance (gm) also becomes large. As a result, the sensitivity increases, and the detection limit is lowered. Furthermore, since the electric field created by the charge of the biological substance 2 can be collected in the semiconductor film 24 from the region immediately above the semiconductor film 24 as well as a wider region through the solid electrolyte membrane 25, the detection target substance can be detected with higher sensitivity, and the detection limit is further lowered. Particularly, when the ion conductivity of the solid electrolyte membrane 25 is 1×10⁻⁸ S/cm or more, these effects are more remarkably observed.

With regard to the sensors 100 and 101 of the present embodiment, when the solid electrolyte membrane 25 is an inorganic solid electrolyte membrane which is a metal oxide including a rare earth element and zirconium (Zr) or a metal oxide including a rare earth element and tantalum (Ta), and in which the amount of carbon (C) is 0.5 atom % or more and 15 atom % or less and the amount of hydrogen (H) is 2 atom % or more and 20 atom % or less, the electric field created by the charge of the biological substance 2 captured to the exposed surface 25a is easily transmitted to the semiconductor film 24. In addition, when the semiconductor film 24 is an inorganic semiconductor film which is a metal oxide including at least indium (In), the amount of change in the electrical characteristics caused by transmission of an electric field becomes large. For this reason, a detection target substance can be detected with higher sensitivity, and the detection limit is further lowered.

In the sensors 100 and 101 of the present embodiment, when probe molecules 1 for capturing the biological substance 2 are immobilized on the exposed surface 25a of the solid electrolyte membrane 25, the selectivity for the detection target substance is further improved.

Since the method for detecting a biological substance according to the present embodiment uses the above-mentioned sensors 100 and 101, it is possible to detect a detection target substance with excellent selectivity, high sensitivity, and a low detection limit.

Thus, embodiments of the present invention have been described in detail above; however, the present invention is not limited to the above-described embodiments, and various modifications and alterations can be made within the scope of the gist of the present invention as described in the claims.

For example, in the present embodiment, a biological substance has been described as an example of the detection target substance; however, the detection target substance is not limited to this. The sensors 100 and 101 of the present embodiment are capable of detecting any detection target substance as long as an electric field is transmitted to the semiconductor film 24 when the detection target substance is captured to the exposed surface 25a of the solid electrolyte membrane 25. As the detection target substance, for example, a charged ionic substance or a substance that generates a charge by being captured to the exposed surface 25a, can be used. The detection target substance may be an organic substance or may be an inorganic substance.

In addition, in the present embodiment, as a configuration for improving the selectivity for a detection target substance to be captured to the exposed surface 25a, a configuration in which probe molecules 1 are immobilized on the exposed surface 25a of the solid electrolyte membrane 25 has been described as an example; however, the configuration is not limited to this. For example, a configuration in which the exposed surface 25a is covered with a permselective membrane, and only the substance that has passed through the permselective membrane is captured to the exposed surface 25a, may be adopted.

In addition, in the present embodiment, a case in which a conductive material film 12 and a solid electrolyte film 13 are laminated in this order between the sensor piece 20 (the first electrode 21, the second electrode 22, and the semiconductor film 24) and the substrate 11, has been described as an example; however, the present invention is not limited thereto. For example, the sensor piece 20 may be disposed directly on the substrate 11. In addition, only the solid electrolyte film 13 may be disposed between the sensor piece 20 and the substrate 11.

### Example 1

### Invention Example 1

### (Deposition of Conductive Material Film)

A SiO₂/Si substrate in which a silicon oxide (SiO₂) film having a thickness of 500 nm was formed on a silicon substrate was prepared. A Ti layer having a thickness of 10 nm and a Pt layer having a thickness of 200 nm were deposited in this order on the silicon oxide film of this SiO₂/Si substrate by a sputtering method to deposit a conductive material film with a two-layer structure of Pt/Ti.

### (Deposition of Solid Electrolyte Film)

Next, a solid electrolyte film was formed by a sol-gel method on the Pt layer of the obtained conductive material film. First, a La_{0.3}Zr_{0.7}O solution as a precursor solution for a solid electrolyte film was applied by a spin coating method to form a precursor layer for a solid electrolyte film. Next, the precursor layer for a solid electrolyte film is preliminarily sintered at 250° C. in an oxygen-containing atmosphere and then mainly sintered at 400° ° C. to form a solid electrolyte film formed of La_{0.3}Zr_{0.7}O having a thickness of 250 nm. The amounts of carbon (C) and hydrogen (H) in the obtained solid electrolyte film were each measured by Rutherford backscattering spectrometry, hydrogen forward scattering spectrometry, and nuclear reaction analysis. As a result, the water amount of the carbon (C) was 2.0 atom %, and the amount of hydrogen (H) was 10.1 atom %. In addition, the ion conductivity of the solid electrolyte film was measured by using an alternating current impedance measuring device (manufactured by BioLogic SAS, SP-300). As a result, the ion conductivity was 6.0×10⁻⁷ S/cm.

The La_{0.3}Zr_{0.7}O solution was prepared as follows.

Lanthanum acetate sesquihydrate and zirconium butoxide were mixed at a ratio of 3:7 (molar ratio), and the obtained mixture was dissolved in propionic acid to a concentration of 0.2 mol/kg in terms of the La_{0.3}Zr_{0.7}O concentration. The obtained mixed solution was refluxed in an oil bath at 110ºC for 30 minutes and then filtered through a membrane filter having a pore size of 0.2 μm to obtain a 0.2 mol/kg La_{0.3}Zr_{0.7}O solution.

### (Deposition of Semiconductor Film)

Next, an In₂O₃ solution as a precursor solution for a semiconductor film was applied on the solid electrolyte film by a spin coating method to form a precursor layer for a semiconductor film. Next, the precursor layer for a semiconductor film was mainly sintered at 250° C. to deposit an inorganic semiconductor film formed of In₂O₃, and then In₂O₃ was processed into a channel shape by dry etching. The size of the semiconductor film was set to 300 μm in width×50 μm in length×20 nm in thickness.

The In₂O₃ solution was prepared as follows. Indium nitrate trihydrate was dissolved in 2-methoxyethanol to a concentration of 0.2 mol/kg in terms of the In₂O₃ concentration. The obtained solution was refluxed in an oil bath at 110° C. for 30 minutes and then filtered through a membrane filter having a pore size of 0.2 μm to obtain an In₂O₃ solution having a concentration of 0.2 mol/kg.

### (Formation of First Electrode and Second Electrode)

A resist film patterned in the shapes of a source electrode and a drain electrode was formed on the solid electrolyte film and the semiconductor film by a photolithography method. Next, an ITO layer having a thickness of 50 nm and an Au layer having a thickness of 500 nm were deposited in this order by a sputtering method on the solid electrolyte film and the semiconductor film, on which the resist film was formed, and then the resist film was removed. A first electrode having a two-layer structure of Au/ITO, a first lead wire, a first terminal, a second electrode, a second lead wire, and a second terminal were formed. The sizes of the first electrode and the second electrode were each set to 320 μm in width×200 μm in length, and the interval between the first electrode and the second electrode was set to 50 μm.

### (Deposition of Solid Electrolyte Membrane)

After forming a resist film on the first terminal and the second terminal, a La_{0.3}Zr_{0.7}O solution as a precursor solution for a solid electrolyte membrane was applied by a spin coating method to form a precursor layer for a solid electrolyte membrane. Next, the precursor layer for a solid electrolyte membrane was preliminarily sintered at 250° C. in an oxygen-containing atmosphere and then mainly sintered at 400° C. to deposit a solid electrolyte membrane formed of La_{0.3}Zr_{0.7}O having a thickness of 20 nm, and then the resist film was removed. The amounts of carbon (C) and hydrogen (H) in the obtained solid electrolyte membrane were each measured by Rutherford backscattering spectrometry, hydrogen forward scattering spectrometry, and nuclear reaction analysis, and the water content of carbon (C) was 2.0 atom %, while the amount of hydrogen (H) was 10.1 atom %.

In this way, a sensor (sensor shown in FIG. 4) in which a first electrode, a second electrode, a semiconductor film, and a solid electrolyte membrane covering those were deposited on a solid electrolyte film of a laminated plate, was produced.

### Comparative Example 1

A sensor was produced in the same manner as in Invention Example 1, except that the solid electrolyte membrane was not deposited.

### Comparative Example 2

A sensor was produced in the same manner as in Invention Example 1, except that a resist membrane was deposited by using a photoresist (TSMR, manufactured by Tokyo Ohka Kogyo Co., Ltd.), instead of the solid electrolyte membrane.

### [Evaluation]

In the sensor produced in Invention Example 1, a holding part (5 mm×10 mm) was formed around the solid electrolyte membrane covering the semiconductor film. In the sensor produced in Comparative Example 1, a holding part (5 mm×10 mm) was formed on the first electrode and the second electrode around the semiconductor film. In the sensor produced in Comparative Example 2, a holding part (5 mm×10 mm) was formed on a resist membrane around the semiconductor film.

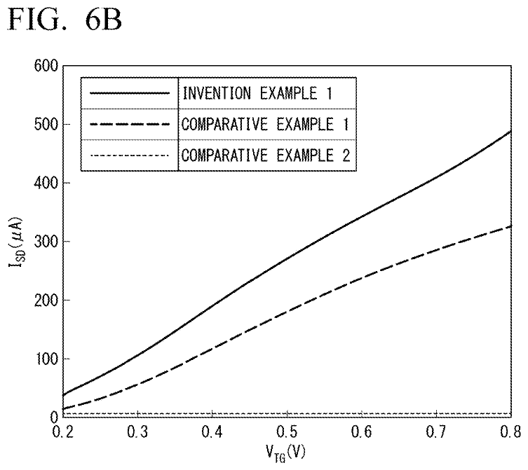

A 0.01× phosphate buffer (PBS) was injected as a conductive liquid into the holding part of the sensor. Next, the third electrode was immersed in the PBS in the holding part of the sensor, and while a voltage V_{TG} was applied between the first electrode and the third electrode, the current value I_{G} flowing through the third electrode and I_{SD} flowing between the first electrode and the second electrode was measured. The voltage V_{IG} was varied from 0.2 V to 0.8 V, and a V_{IG}-I_{G} curve and a V_{IG}-I_{SD} curve were obtained. The V_{IG}-I_{G} curve is shown in FIG. 6A, and the V_{IG}-I_{SD} curve is shown in FIG. 6B.

From the graph of FIG. 6A, it can be seen that in the sensor of Invention Example 1 in which the first electrode, the second electrode, and the semiconductor film are covered with a solid electrolyte membrane, and the sensor of Comparative Example 2 in which the first electrode, the second electrode, and the semiconductor film are covered with a resist membrane, an amount of change in the current value In caused by an increase in the voltage V_{TG} can be hardly seen. In contrast, it can be seen that in the sensor of Comparative Example 1 in which the first electrode, the second electrode, and the semiconductor film are not covered with a solid electrolyte membrane, the current value I_{G} increases even due to an increase in the voltage V_{TG}. The current value I_{G} is a leakage current value obtained by the current flowing from the first electrode to the second electrode leaking to the third electrode. Therefore, from the graph of FIG. 6A, it can be seen that in the sensor of Invention Example 1, the generation of leakage current is markedly suppressed as compared with the sensor of Comparative Example 1.

In addition, from the graph of FIG. 6B, it can be seen that in the sensor of Invention Example 1, the current value I_{SD} at each of the voltages V_{TG} is large, and the amount of change in the current value I_{SD} caused by an increase in the voltage V_{IG} is large, as compared with the sensor of Comparative Example 1. For example, the current value I_{SD} at the time of setting the voltage V_{TG} to 0.8 V is 310 μA for the sensor of Comparative Example 1, whereas the current value I_{SD} of the sensor of Invention Example 1 is 480 μA, which is about 1.5 times higher. Therefore, the sensor of Invention Example 1 has a higher gm and is capable of measurement with higher sensitivity, as compared with the sensor of Comparative Example 1. On the other hand, since the sensor of Comparative Example 2 has a lower current value I_{SD} at each voltage V_{TG} as compared with the sensor of Comparative Example 1, it can be seen that the sensor of Comparative Example 2 has a lower gm and lower sensitivity as compared with the sensor of Comparative Example 1.

### Invention Example 2

Escherichia coli was detected as follows by using the sensor produced in Invention Example 1.

### (Immobilization of Probe DNA)

A wall part was provided around the solid electrolyte membrane covering the semiconductor film of the sensor to form a holding part (5 mm×10 mm). Next, a DNA complementary to a portion of 16s-rRNA of Escherichia coli was immobilized as a probe DNA (probe molecule) on the exposed surface within the holding part of the sensor. The sequence of the probe DNA is shown in the following Table 1.

TABLE 1
AGCCCGGGGATTTCACATCTGACTTA
(Remarks) The left-hand side is the 5′-terminal.

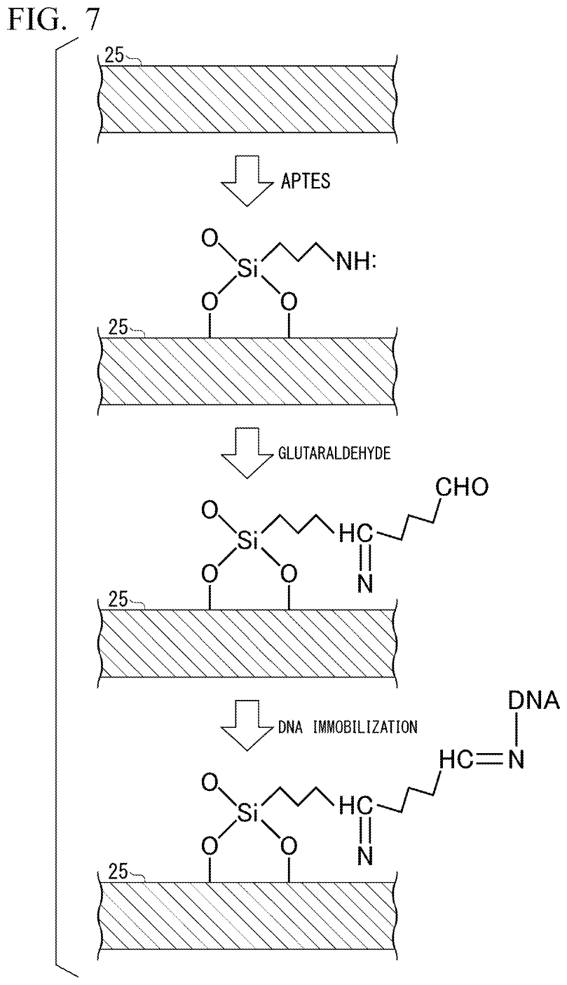

The probe DNA was immobilized within the holding part of the sensor according to the procedure shown in FIG. 7.

First, 3-aminopropyltriethoxysilane (APTES) was bonded to the area within the holding part (solid electrolyte membrane 25). Next, an amino group of APTES and one aldehyde group of glutaraldehyde were reacted to bind APTES to glutaraldehyde. Then, finally, the other aldehyde group of glutaraldehyde and the probe DNA were reacted to immobilize 100 nmol/L of the probe DNA.

### (Preparation of Test Liquid)

Escherichia coli and an aqueous solution of sodium dodecyl sulfate (SDS) at a concentration of 1% by mass were mixed to prepare a test liquid. In Escherichia coli in the test liquid, the cell wall and nucleolytic enzymes were disrupted, and DNA and mRNA were released. As the test liquid, a solution having an Escherichia coli concentration of 10.1×10⁴ cells/uL was prepared.

(Detection of Escherichia coli)

2 μL of the test liquid was dropped onto the holding part of the sensor, and the sensor was incubated for 5 minutes at room temperature. Next, the test liquid was removed from the holding part of the sensor, and pure water was injected into the holding part to wash the exposed surface of the solid electrolyte membrane. Thereafter, a 0.01× phosphate buffer (PBS) was injected as a conductive liquid into the holding part to replace the test liquid with the 0.01×PBS.

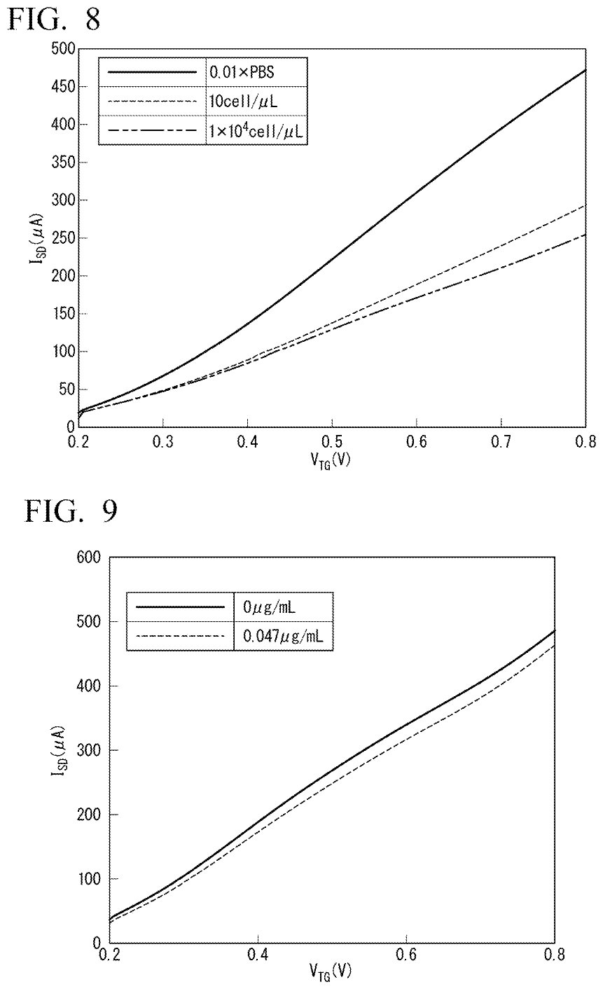

Next, the third electrode was immersed in the PBS in the holding part of the sensor, and while a voltage V_{TG} was applied between the first electrode and the third electrode, the I_{SD} flowing between the first electrode and the second electrode was measured. The voltage V_{TG} was varied from 0.2 V to 0.8 V, and a V_{TG}-I_{SD} curve was obtained. The results are shown in FIG. 8. In addition, a 0.01×PBS was injected into the holding part of the sensor without dropping the test liquid thereon, and a V_{IG}-I_{SD} curve was obtained in the same manner. The results are shown in FIG. 8.

It can be seen from the graph of FIG. 8 that the amount of change in the current value I_{SD} caused by an increase in the voltage V_{TG} varies depending on the number of cells of Escherichia coli in the test liquid. It can be seen that as compared with a case where a 0.01×PBS including no Escherichia coli was used, when a test liquid including a number of cells of Escherichia coli was used, the amount of change in the current value I_{SD} caused by an increase in the voltage V_{TG} was remarkably reduced.

### Invention Example 3

The measurement sensitivity for DNA was evaluated as follows by using the sensor produced in Invention Example 1. In the same manner as in Invention Example 2, a DNA complementary to a portion of 16s-rRNA of Escherichia coli was immobilized as a probe DNA on the exposed surface within the holding part of the sensor. The area of the exposed surface in the holding part was 21 mm² (7 mm×3 mm). Then, 2 μL of a test liquid having an Escherichia coli DNA concentration of 0.047 μg/mL was dropped onto the holding part of the sensor, and the sensor was incubated at room temperature for 10 minutes. Next, the test liquid was removed from the holding part of the sensor, and pure water was injected into the holding part to wash the exposed surface of the solid electrolyte membrane. Thereafter, a 0.01×PBS was injected as a conductive liquid into the holding part to replace the test liquid with the 0.01×PBS.

Next, in the same manner as in Invention Example 2, the third electrode was immersed in PBS in the holding part of the sensor, and while a voltage V_{TG} was applied between the first electrode and the third electrode, the I_{SD} flowing between the first electrode and the second electrode was measured. Similarly, a test was performed by using a test liquid in which the concentration of Escherichia coli DNA was 0 μg/mL (blank). The results are shown in FIG. 9.

From the graph of FIG. 9, since there was a difference in the value of I_{SD} at each V_{TG} between a case where a test liquid having a concentration of Escherichia coli DNA of 0 μg/mL (blank) was used and a case where a test liquid having a concentration of Escherichia coli DNA of 0.047 μg/mL was used, it can be seen that the sensor produced in Invention Example 1 has a detection limit for Escherichia coli DNA that is lower than 0.047 μg/mL.

### INDUSTRIAL APPLICABILITY

The present invention can provide a sensor that has excellent selectivity to a detection target substance and has high sensitivity and a low detection limit, and a method for detecting a biological substance.

### REFERENCE SIGNS LIST

1: Probe molecule
2: Biological substance
11: Substrate
12: Conductive material film
13: Solid electrolyte film
21: First electrode
22: Second electrode
23: Third electrode
24: Semiconductor film
25: Solid electrolyte membrane
25a: Exposed surface
30: Holding part
31: First voltage supply part
32: Second voltage supply part
100, 101: Sensor

1. A sensor comprising:
a first electrode;
a second electrode;
a third electrode;
a semiconductor film configured to connect the first electrode and the second electrode; and
a solid electrolyte membrane configured to cover the first electrode, the second electrode,
and the semiconductor film,
wherein the solid electrolyte membrane has an exposed surface exposed to an outside, and
the third electrode is configured to be disposed at a position where, when the exposed surface of the solid electrolyte membrane is in contact with a conductive liquid, it is possible to apply an electric field to the exposed surface of the solid electrolyte membrane through the conductive liquid.

2. The sensor according to claim 1,
wherein the first electrode, the second electrode, and the semiconductor film are arranged on one substrate.

3. The sensor according to claim 2,
wherein a conductive material film and a solid electrolyte film are laminated between the substrate and the first electrode, the second electrode, as well as the semiconductor film, and
the first electrode, the second electrode, and the semiconductor film are arranged on the solid electrolyte film.

4. The sensor according to claim 2,
wherein the third electrode is further disposed on the substrate.

5. The sensor according to claim 1,
wherein the solid electrolyte membrane has an ion conductivity of 1×10⁻⁸ S/cm or more.

6. The sensor according to claim 1,
wherein the solid electrolyte membrane is an inorganic solid electrolyte membrane which is a metal oxide including a rare earth element and zirconium (Zr) or a metal oxide including a rare earth element and tantalum (Ta), and in which an amount of carbon (C) is 0.5 atom % or more and 15 atom % or less and an amount of hydrogen (H) is 2 atom % or more and 20 atom % or less, and
the semiconductor film is an inorganic semiconductor film which is a metal oxide including at least indium (In).

7. The sensor according to claim 1,
wherein probe molecules intended for capturing a biological substance are immobilized on the exposed surface of the solid electrolyte membrane.

8. The sensor according to claim 1, further comprising a holding part intended for holding the conductive liquid around the exposed surface of the solid electrolyte membrane.

9. A method for detecting a biological substance using the sensor according to claim 1, the method comprising:
a step of supplying a test liquid including a biological substance to the exposed surface of the solid electrolyte membrane to capture the biological substance to the exposed surface;
a step of replacing the test liquid with the conductive liquid;
a step of applying a voltage between the third electrode and the first electrode, and at the same time, measuring a current between the first electrode and the second electrode; and
a step of acquiring an amount of the biological substance in the test liquid based on the voltage and the current.

10. The sensor according to claim 3,
wherein the third electrode is further disposed on the substrate.

11. The sensor according to claim 2,
wherein the solid electrolyte membrane has an ion conductivity of 1×10⁻⁸ S/cm or more.

12. The sensor according to claim 3,
wherein the solid electrolyte membrane has an ion conductivity of 1×10⁻⁸ S/cm or more.

13. The sensor according to claim 2,
wherein the solid electrolyte membrane is an inorganic solid electrolyte membrane which is a metal oxide including a rare earth element and zirconium (Zr) or a metal oxide including a rare earth element and tantalum (Ta), and in which an amount of carbon (C) is 0.5 atom % or more and 15 atom % or less and an amount of hydrogen (H) is 2 atom % or more and 20 atom % or less, and
the semiconductor film is an inorganic semiconductor film which is a metal oxide including at least indium (In).

14. The sensor according to claim 3,
wherein the solid electrolyte membrane is an inorganic solid electrolyte membrane which is a metal oxide including a rare earth element and zirconium (Zr) or a metal oxide including a rare earth element and tantalum (Ta), and in which an amount of carbon (C) is 0.5 atom % or more and 15 atom % or less and an amount of hydrogen (H) is 2 atom % or more and 20 atom % or less, and
the semiconductor film is an inorganic semiconductor film which is a metal oxide including at least indium (In).

15. The sensor according to claim 2,
wherein probe molecules intended for capturing a biological substance are immobilized on the exposed surface of the solid electrolyte membrane.

16. The sensor according to claim 3,
wherein probe molecules intended for capturing a biological substance are immobilized on the exposed surface of the solid electrolyte membrane.

17. The sensor according to claim 2, further comprising a holding part intended for holding the conductive liquid around the exposed surface of the solid electrolyte membrane.

18. The sensor according to claim 3, further comprising a holding part intended for holding the conductive liquid around the exposed surface of the solid electrolyte membrane.

19. A method for detecting a biological substance using the sensor according to claim 2, the method comprising:
a step of supplying a test liquid including a biological substance to the exposed surface of the solid electrolyte membrane to capture the biological substance to the exposed surface;
a step of replacing the test liquid with the conductive liquid;
a step of applying a voltage between the third electrode and the first electrode, and at the same time, measuring a current between the first electrode and the second electrode; and
a step of acquiring an amount of the biological substance in the test liquid based on the voltage and the current.

20. A method for detecting a biological substance using the sensor according to claim 3, the method comprising:
a step of supplying a test liquid including a biological substance to the exposed surface of the solid electrolyte membrane to capture the biological substance to the exposed surface;
a step of replacing the test liquid with the conductive liquid;
a step of applying a voltage between the third electrode and the first electrode, and at the same time, measuring a current between the first electrode and the second electrode; and
a step of acquiring an amount of the biological substance in the test liquid based on the voltage and the current.
